(19) United States
(12) Patent Application Publication
(10) Pub. No.: US 2007/0122809 A1
(43) Pub. Date: May 31, 2007

(54) Nucleic acid isolation

(75) Inventors: Anthony Stevenson, Kent (GB); Andrea Potts, Kent (GB); Declan Donovan, Kent (GB); Matthew J. Baker, Kent (GB)

Correspondence Address: VINSON & ELKINS, L.L.P., 1001 FANNIN STREET, 2300 FIRST CITY TOWER, HOUSTON, TX, 77002-6760, US

(73) Assignee: Invitrogen Corporation

(21) Appl. No.: 10/565,951

(22) PCT Filed: Jul. 21, 2004
(86) PCT No.: PCT/GB04/03165

§ 371 Date: Aug. 7, 2006

(30) Foreign Application Priority Data
Jul. 21, 2003 (GB) 0317044.6

(51) Int. Cl. C12Q 1/68; C07H 21/04

(52) U.S. Cl. 435/6; 536/25.4

(57) ABSTRACT

The invention provides materials and methods for purifying nucleic acids from a biological sample held in a container (30) such as a centrifuge tube. A filtration unit (1) having a filter (7, 9) and a filtrate chamber (5) is placed within the container (30). Preferably the exterior of the filtration unit (1) engages sealingly with the interior surface of the container (30). Relative movement between the container (30) and the filtration unit (1) causes the sample to pass through the filter (7, 9) into the filtrate chamber (5), where it can be further manipulated. In preferred embodiments the filtrate is then contacted with a charge-switch nucleic acid binding agent in order to purify the nucleic acid further.

### FIELD OF THE INVENTION

The present invention relates to methods for extracting nucleic acids from samples and products useful in carrying out these methods.

### BACKGROUND TO THE INVENTION

There is a very large demand for DNA analysis for a range of purposes and this has lead to the requirement for quick, safe, high throughput methods for the isolation and purification of DNA and other nucleic acids.

Samples for use for DNA identification or analysis can be taken from a wide range of sources such as biological material such as animal and plant cells, faeces, tissue etc. also samples can be taken from soil, foodstuffs, water etc.

Numerous methods exist for the extraction of DNA, including the use of phenol/chloroform, salting out, the use of chaotropic salts and silica resins, the use of affinity resins, ion exchange chromatography, the use of magnetic beads, and use of charge switch materials. Methods are described in U.S. Pat. Nos. 5,057,426, 4,923,978, EP 512767 A, EP 0515484 A, WO95/13368, WO97/10331, WO96/18731 and WO02/48164.

Many of these techniques require the nucleic acid to be present in a clarified solution, substantially free of insoluble material. However nucleic acid purification procedures often involve cell lysis and protein precipitation steps prior to the actual isolation of the nucleic acid. For this reason, many protocols require one or more centrifugation steps, each often followed by the removal of supernatant containing nucleic acid. This can be time consuming, may reduce final yields of nucleic acid due to incomplete recovery of supernatant at each step, and can also increase the cost of nucleic acid isolation as the amount of time and consumables required to perform the procedure increases.

### SUMMARY OF THE INVENTION

The present invention aims to address some of the problems associated with prior art protocols.

The invention relates broadly to the use of filtration units for filtering samples containing nucleic acid without the need first to remove the sample from its container.

Thus the present invention provides a method of purifying nucleic acid from a sample solution in a container, comprising causing relative movement between the container and a filtration unit, the filtration unit being disposed at least partly within the container and having a filter and a filtrate chamber, whereby the sample is made to pass through the filter into the filtrate chamber.

In preferred embodiments the filtration unit has a “plunger” configuration which enables it to be inserted into a tube containing a sample such that the sample is forced through the filter and into the filtrate chamber, leaving insoluble material such as precipitated proteins and cell debris in the tube or on the filter. The method may therefore include the step of inserting the filtration unit into the container.

Preferably an exterior surface of the filtration unit forms a seal with an interior surface of the container. This prevents the sample being forced between the sides of the container and filtration unit instead of through the filter, preventing sample loss and also ensuring that as much of the sample as possible is filtered and retained in the filtrate chamber. However it is essential that the filtration unit and container remain able to move relative to one another once sealing contact has been made. Typically the filtration unit comprises a seal on an exterior surface thereof adapted to make sealing contact with a particular (often standard) type of container. Examples of such standard containers include standard disposable conical-bottomed 15 ml or 50 ml centrifuge tubes of the type in which biological purification procedures are often performed, as available from manufacturers of laboratory ware and consumables such as Becton Dickinson under the trade mark “Falcon”.

The filtration unit may have a tip shaped to be complementary to the bottom of such a standard tube. Thus for use in round-bottomed tubes, the filtration unit may have a rounded tip, while for use in conical bottomed tubes such as those described above the tip may have a tapered, conical or frustoconical cross-section. Shaping the tip in this way enables the filtration unit to be inserted as far as possible into a container holding a sample, allowing filtration and recovery of as much of the sample as possible.

The end of the filtration unit remote from the tip is typically open to allow easy access to the filtrate. However this end of the unit may be sealable, e.g. by means of a cap, to prevent loss of filtrate, or to facilitate storage thereof in the filtrate chamber. The filtration unit may be supplied in sealed form, which may be particularly appropriate if the unit has been pre-sterilised, as it enables the interior of the unit to remain sterile.

The filtration unit may also comprise external projections adapted to reduce lateral movement of the filtration unit during and/or after insertion into a container holding a sample. These projections may have any suitable form, including longitudinal vanes, transverse flanges, etc. These projections (especially longitudinal projections) may also serve to strengthen the body of the filtration unit against deformation as it is pushed into a container.

The filtration unit may comprise at least upstream and downstream filters in series. The upstream filter, i.e. that first contacted by the sample, may be regarded as a pre-filter. Selectivity of either or both of the filters may rely on particle size or weight (e.g. via filter pore size and/or molecular weight cut-off), and/or on selective binding characteristics of the filter material. Thus the filter may have a higher affinity for certain components of the sample than for others. Such filters may be regarded as “active” filters.

The upstream filter preferably has a higher size exclusion threshold than the downstream filter, i.e. the filter closest to the filtrate chamber. By size exclusion threshold is meant the maximum size of particle which can pass through the filter. Thus the upstream filter may allow particles to pass through it which are retained by the downstream filter. Relatively large insoluble components of the sample may be retained by the coarser upstream filter and so prevented from blocking the finer downstream filter, while smaller insoluble components which pass through the upstream filter are retained by the downstream filter. The upstream filter may be formed from a plug of fibrous material (e.g. sintered or extruded plastics material, cellulose, glass fibre, plastics mesh etc.) and may not have a single defined pore size, but may tend to trap material likely to block the finer downstream filter. It may have an average pore size greater than that of the downstream filter. For example, the upstream filter may have an average pore size of 5 to 500 micrometers, while the downstream filter may have an average pore size of 0.1 to 5 micrometers. The upstream filter (or pre-filter) may be housed within the tip of the filtration unit.

Either or both filters, but preferably the upstream filter, may selectively retain particular molecular types of high affinity for particular molecular types, e.g. for proteins or carbohydrates. For example, a filter carrying hydrophobic surface groups (e.g. phenyl groups or other suitable hydrophobic groups) would be expected to bind non-specifically to proteins, as would a filter carrying negatively charged surface groups (which may have a particularly high affinity for the positively charged proteins often found associated with nucleic acids, such as histones, protamines and bacterial nucleic acid binding proteins). Both of these filter types would be expected to bind proteins in preference to relatively hydrophilic, negatively charged nucleic acids. The filter material may also be derivatised with specific binding agents in order to increase the amount of particular identified contaminants retained by the filter. For example derivatisation with lectins would be expected to increase the amount of carbohydrate retained by the filter, while derivatisation with antibodies would increase the amount of their cognate antigen(s) retained. A filter may carry one or more of the above-mentioned groups or derivatisations, or any others which the skilled person may find appropriate depending upon the particular application of the method of the invention.

The filtration unit may be used to filter any sample containing nucleic acid. Thus the sample may be prepared by any appropriate means. Typically to prepare a sample from cellular material requires a cell lysis step to release nucleic acids, which can be performed by means of known lysing agents and methods, such as contacting with ionic and non ionic detergents, hypotonic solutions of salts, proteases, chaotropic agents, solvents, using pH changes or heat. A method of lysing cells to isolate nucleic acid is described in WO 96/00228.

When the cells are obtained from blood, the blood can optionally be diluted with water or other diluent in order to make it easier to manipulate and to process. Preparation of samples from solid tissues may require enzymatic digestion to release cells from extrcellular matrices etc. After cell lysis, the sample may be treated with reagents to remove components other than nucleic acids from the solution; e.g. proteins may be precipitated by addition of potassium ions to a lysate containing a detergent such as SDS. Such precipitated components and cell debris will be removed from the sample in the methods of the present invention by the filter or filters of the filtration unit, to give a clarified filtrate.

However the methods of the invention are equally applicable to further purification of cell-free solutions containing nucleic acid. For example, enriched extractions of nucleic acid such as crude preparations of nuclei, chromatin or DNA can be easily isolated using commercially available magnetic bead technology, and further purified using the methods of the present invention.

By appropriate prior preparation of the sample, any nucleic acid can be purified by the methods of the invention, including genomic DNA (eukaryotic, prokaryotic or viral), episomal DNA such as plasmids, cDNA, or RNA (including mRNA, rRNA, tRNA), in either single stranded or double stranded form. Undesired nucleic acid types may be selectively precipitated from the sample before filtration by techniques well known to those skilled in the art, e.g. by suitable adjustment of salt concentrations, optionally in conjunction with binding agents which bind one nucleic acid type in preference to another under the relevant conditions (see below).

Depending upon the method used to prepare the sample, the filtrate may or may not require further purification. If the filtrate contains low salt or non-inhibitory substances then the filtrate may, for example, be added directly to a PCR or other analytical reaction. Otherwise, the method may comprise the step of further purifying nucleic acid from the filtrate.

In preferred embodiments, the further purification involves contacting the filtrate with a nucleic acid binding agent. The binding agent may be in the solid or liquid phase, but solid phases are preferred. Any suitable binding agent may be used, such as silica resins, plastic frits or tips, membranes, affinity resins, and magnetic beads. These solid phases may also be used in conjunction with concentrating reagents such as alcohols, salts or polyethylene oxides.

Particularly preferred is the use of the use of a charge switch material as described in our copending applications European Patent Application No: 98957019.7, U.S. patent application Ser. No. 09/736,632 and WO 02/48164. In preferred embodiments, the target nucleic acid is purified using a charge switch material, e.g. present on a solid phase, a pipette tip, beads (especially magnetic beads), a porous membrane, a frit, a sinter, a probe or dipstick, a tube (PCR tube, Eppendorf tube) or a microarray. Charge switch materials may be solid phases or soluble. They comprise ionisable groups such that they are capable of binding nucleic acid at a first pH (typically between pH 5.0 and 9.0), and then releasing it at a second higher pH (typically less than 10.5). Examples of solid phase charge switch materials are those in which (1) the ionisable groups are separately immobilised on a solid support by covalent or ionic bonding or by adsorption, (2) the ionisable groups are separately attached to a polymer, said polymer being immobilised on a solid support by covalent or ionic bonding or by adsorption, (3) the ionisable groups are polymerised, optionally by means of cross-linking reagents, and the polymer is immobilised on a solid support by covalent or ionic bonding or by adsorption. By way of example, the charged groups in the charge switch material may be provided by a biological buffer (e.g. Tris, Bis-Tris, polyTris or poly Bis-Tris), a polyhydroxylated amine, a detergent or surfactant, a carbohydrate, a nucleic acid base, a heterocyclic nitrogen-containing compound, a monoamine, a biological dye, or a negatively ionisable group, the pKa of which is between about 3.0 and 7.0 and a metal oxide which is positively charged at said first pH, and optionally also at said second pH.

Generally the charge switch material will change charge because of a change in charge on a positively ionisable group from positive to less positive or neutral, as the pH is increased in a range spanning or close to the pKa of the positively ionisable group. This may also be combined with a change of charge on a negatively ionisable group from neutral or less negative to more negative. In an alternative embodiment (described below), however, the charge switch material comprises a material which is positively charged at both pH values (such as a metal oxide) and a negatively ionisable group, the charge of which becomes more negative as the pH is increased in a range spanning or close to its pKa.

The charge switch material may comprise an ionisable group having a pKa between about 3 and 9. For positively ionisable groups, the pKa is more preferably at least about 4.5, 5.0, 5.5, 6.0 or 6.5 and/or at most about 8.5, 8.0, 7.5 or 7.0. A particularly preferred pKa for a positively ionisable group is between about 5 and 8; even more preferred is a pKa between about 6.0 and 7.0, more preferably between about 6.5 and 7.0. The pKa for negatively ionisable groups is preferably between about 3 and 7, still more preferably between about 4 and 6, further preferably approximately at the pH at which it is desired to bind nucleic acid.

Materials having more than one pKa value (e.g. having different ionisable groups), or combinations of materials having different pKa values, may also be suitable for use as charge switch materials in accordance with the invention, provided that at a first (lower) pH the material(s) possess(es) a positive charge and that at a higher pH the charge is less positive, neutral or negative.

Generally a charge switch will be achieved by changing the pH from a value below to a value above the pKa of the or an ionisable group. However, it will be appreciated that when the pH is the same as the pKa value of a particular ionisable group, 50% of the individual ionisable groups will be charged and 50% neutral. Therefore, charge switch effects can also be achieved by changing the pH in a range close to, but not spanning, the pKa of an ionisable group. For example, at the pKa of a negatively ionisable group, such as a carboxy group (pKa typically around 4), 50% of such groups will be in the ionised form (e.g. COO⁻) and 50% in the neutral form (e.g. COOH). As the pH increases, an increasing proportion of the groups will be in the negative form.

Preferably the binding step is carried out at a pH of below the pKa of the ionisable group, or (though this is not preferred) within about 1 pH unit above the pKa. Generally the releasing step is carried out at a pH above the pKa of the ionisable group, preferably at a pH between 1 and 3 pH units above the pKa.

Charge switch-type binding agents may also be used in the preparation of the sample before filtration as described above. For example, these agents can selectively bind different types of nucleic acids according to size, and whether they are single or double stranded, by adjustment of the salt concentration. Such techniques are well known to those skilled in the art.

The binding agent may be added to the filtrate chamber, the filtrate may be removed from the filtrate chamber prior to contact with the binding agent, or the filtrate may be contacted with the binding agent as it is removed from the filtrate chamber.

In preferred embodiments the filtrate is drawn through a column of solid phase binding agent containing or comprising the nucleic acid binding agent. This may be achieved by any appropriate means, typically by applying a pressure differential across the column by means of a vacuum pump or a syringe, although a syringe may be particularly convenient. Thus the desired nucleic acid is retained on the column while any other impurities pass through the column (e.g. into the body of the syringe) and can be discarded.

In preferred embodiments the column of solid phase binding material is in the form of a cartridge, i.e. a pre-packed column in a physically robust housing which can be conveniently handled and stored (e.g. at ambient temperature for long periods of time). Preferably the cartridge housing is adapted to be easily connected to standard laboratory equipment for convenient operation. For example the cartridge may be specifically adapted for connection to a syringe; e.g. it may comprise a Luer-Lok type fitting. The cartridge may also comprise an elongate tip to facilitate efficient recovery of the filtrate from the filtrate chamber.

However the nucleic acid is contacted with the binding agent, the method may further comprise the steps of washing the binding agent to remove impurities, and/or eluting the bound nucleic acid. The skilled person will be capable of designing suitable wash and elution protocols depending upon the particular binding agent used.

Other methods of nucleic acid purification may also be used to further isolate nucleic acid from the filtrate, such as buffer exchange (e.g. by dialysis or ultrafiltration) or precipitation (e.g. with an alcohol, such as ethanol or isopropyl alcohol, or with polyethylene oxide).

The present invention further provides a filtration unit (e.g. of the “plunger” type) as described above for use in a method of nucleic acid isolation. The filtration unit may comprise a barrel and a tip, with a continuous bore therethrough and a filter disposed within the bore. As described above, the filtration unit is preferably sized for insertion into a standard centrifuge tube or other standard laboratory reaction tube, and is preferably adapted to make sealing contact with an interior surface thereof.

As described above, the tip may be shaped to be complementary to the interior contour of a standard laboratory tube.

Preferably the filtration unit comprises at least upstream and downstream filters as described above.

The filtration unit may comprise a seal suitably sized for sealing engagement with the interior wall of the container. The seal may comprise an annular flange, which may in turn comprise a substantially rigid inner portion and a flexible outer portion which in use engages the wall of the tube. However other possible seal configurations, such as a simple O-ring type seal, will be apparent to the skilled person.

The exterior surface of the barrel preferably carries projections adapted to reduce lateral movement of the plunger within a centrifuge tube. In preferred embodiments the projections are longitudinal vanes or transverse flanges.

The present invention also provides a kit comprising a filtration unit as described herein, in combination with a nucleic acid binding agent. Preferably the nucleic acid binding agent is pre-packed in a column format, e.g. as a cartridge as described supra.

The nucleic acid binding agent is preferably a charge switch agent. The cartridge may adapted for connection to a syringe and additionally or alternatively may comprise an elongate tip for drawing a sample though the nucleic acid binding agent.

The kit may further comprise a tube capable of sealing engagement with the filtration unit.

The kit may further comprise reagents for use in isolation of nucleic acids. For example, the kit may comprise reagents for cell lysis, protein precipitation, nucleic acid precipitation, washing of nucleic acid binding agents and elution from nucleic acid binding agents.

### BRIEF DESCRIPTION OF THE FIGURES

FIG. 1 shows a cross section of a plunger-type filteration unit for use in nucleic acid purification.

FIG. 2 shows a schematic diagram of the filtration unit of FIG. 1 in use.

### EXAMPLES

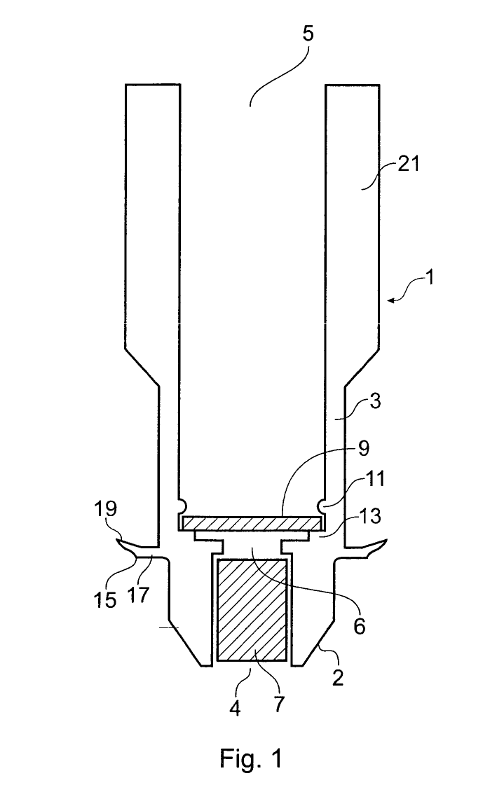

As shown in FIG. 1, a filtration unit 1 for use in nucleic acid isolation comprises an elongate cylindrical barrel 3 and a tapered tip 2 which has a frustoconical cross section, complementary to the interior contour of a standard conical-bottomed 50 ml “Falcon™” centrifuge tube as available from Becton Dickinson and other suppliers of laboratory consumables. The filtration unit has a continuous bore comprising a filtrate chamber 5 located within the barrel and a pre-filter chamber 4 within the tip 2, separated by a narrow neck 6.

Pre-filter chamber 4 contains a relatively coarse pre-filter 7 formed from a plug of extruded plastic. At the bottom of filtrate chamber 5 is a fine filter 9, having a pore size of 5 micrometers, held between an annular snap ring 11 and an annular shoulder 13.

At the bottom of barrel 3, approximately level with neck 6, the exterior surface of the filtration unit 1 carries a seal 15, which consists of an annular flange having a rigid inner portion 17 and an outer flexible portion 19.

At its other end, the barrel 3 carries four equally spaced longitudinal vanes 21 which run approximately half the length of barrel 3 towards the tip 2.

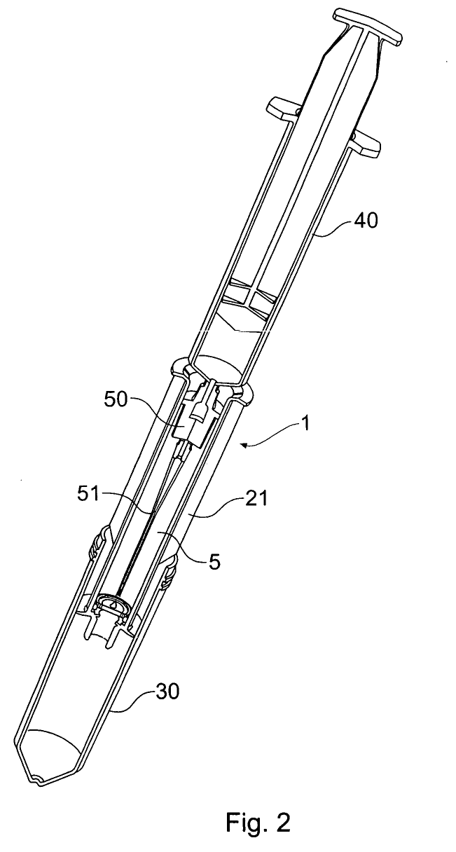

The practical use of filtration unit 1 will now be described by reference to FIG. 2.

A sample solution is prepared in a standard 50 ml centrifuge tube 30 by enzymatic digestion of tissue from which it is desired to extract nucleic acid. For example, a pellet of mammalian cells may be treated with 1% SDS, 10 mM EDTA, 10 mM Tris HCl pH8 and proteinase K for 30 minutes at room temperature. The proteins are then precipitated by adding 1.5M potassium acetate, pH4.

Filtration unit 1 is then inserted into the top of tube 30 and pushed downwardly to contact the sample, which is forced through pre-filter 7 and fine filter 9 (not shown in FIG. 2) into the filtrate chamber 5. The shaping of the tip 2 allows the filtration unit to be pressed tight down into the tube 30 resulting in maximal recovery of sample. Vanes 21 prevent substantial lateral movement of the filtration unit 1 as it is pushed into tube 30.

Insoluble contaminants are retained on the two filters 7 and 9, leaving a clarified solution in the filtrate chamber.

The filtrate is then drawn up from the filtrate chamber 5 into a syringe 40 through a cartridge 50 which contains a charge-switch nucleic acid binding resin. The cartridge has an elongate tip 51 which helps the entire filtrate to be recovered from the filtrate chamber 5.

Thus nucleic acids in the sample remain bound to the resin, while other components of the sample pass into the syringe and can be discarded, e.g. by simply expelling the solution through the cartridge 50. This may enable the resin to bind any nucleic acid not bound on the original pass through the cartridge. The resin may then be washed if desired and the nucleic acid eluted.

All of the references mentioned herein are expressly incorporated by reference.

1. A method of purifying nucleic acid from a sample solution in a container, comprising causing relative movement between the container and a filtration unit, the filtration unit being disposed at least partly within the container and having a filter and a filtrate chamber, whereby the sample is made to pass through the filter into the filtrate chamber.

2. A method according to claim 1 wherein an exterior surface of the filtration unit forms a seal with an interior surface of the container.

3. A method according to claim 1 or claim 2 wherein the filtration unit comprises at least upstream and downstream filters in series.

4. A method according to claim 3 wherein the upstream filter has a higher size exclusion threshold than the downstream filter.

5. A method according to any one of claims 1 to 4 wherein the container is a centrifuge tube.

6. A method according to any one of the preceding claims comprising further purifying nucleic acid from the filtrate.

7. A method according to claim 6 comprising contacting the filtrate with a nucleic acid binding agent.

8. A method according to claim 7 wherein the nucleic acid binding agent is a charge-switch agent.

9. A method according to claim 7 or claim 8 comprising passing the filtrate through a cartridge comprising said nucleic acid binding agent.

10. A method according to claim 9 wherein the filtrate is drawn through said cartridge by suction.

11. A method according to claim 10 wherein the filtrate is drawn through the cartridge by a syringe.

12. A method according to any one of claims 7 to 11 further comprising washing the nucleic acid binding agent.

13. A method according to any one of claims 7 to 12 further comprising eluting the nucleic acid from the nucleic acid binding agent.

14. A method according to claim 6 comprising further purifying nucleic acid from the filtrate by buffer exchange or precipitation.

15. A method according to claim 14 wherein said buffer exchange comprises dialysis or ultrafiltration.

16. A filtration unit for use in a method of nucleic acid isolation, comprising a barrel having a continuous bore therethrough and a filter disposed within the bore.

17. A filtration unit according to claim 16, comprising a seal adapted to make sealing engagement with the interior wall of a standard centrifuge tube.

18. A filtration unit according to claim 17 wherein the seal comprises an annular flange.

19. A filtration unit according to claim 18 wherein the annular flange has a substantially rigid inner portion and a flexible outer portion.

20. A filtration unit according to any one of claims 16 to 19 comprising two filters of different exclusion thresholds disposed within the bore.

21. A filtration unit according to any one of claims 16 to 20 wherein the exterior surface of the barrel carries projections adapted to reduce lateral movement of the plunger within a centrifuge tube.

22. A filtration unit according to claim 21 wherein said projections are longitudinal vanes or transverse flanges.

23. A filtration unit according to any one of claims 16 to 22 comprising a tip having a shape complementary to the interior contour of the bottom of a standard centrifuge tube.

24. A filtration unit according to claim 23 wherein the tip has a tapered or rounded cross section.

25. A kit comprising a filtration unit according to any one of claims 16 to 24 and a nucleic acid binding agent.

26. A kit according to claim 25 wherein the nucleic acid binding agent is in pre-packed column format.

27. A kit according to claim 26 wherein the column is in the form of a cartridge adapted for connection to a syringe.

28. A kit according to claim 27 wherein the cartridge comprises an elongate tip for drawing a sample though the nucleic acid binding agent.

29. A kit according to any one of claims 25 to 28 wherein the nucleic acid binding agent is a charge switch agent.

30. A kit according to any one of claims 25 to 29 comprising a tube having an internal surface capable of making sealing contact with the filtration unit.

31. A kit according to any one of claims 25 to 30 further comprising one or more wash and/or elution reagents.

32. A kit according to any one of claims 25 to 31 further comprising a sample extraction buffer reagent and/or a protein precipitation reagent.
